SEQUENCE LISTING (7 sequences)

SEQ ID NO: 1 - DNA, 48 bp, Artificial Sequence
  Synthetic construct
tctggtctggtgccacgcggttctgccatggctgatatcggatccgaa

SEQ ID NO: 2 - DNA, 48 bp, Artificial Sequence
  Synthetic construct
ttcggatccgatatcagccatggcagaaccgcgtggcaccagaccaga

SEQ ID NO: 3 - DNA, 5900 bp, Artificial Sequence
  Synthetic construct
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatggccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 4 - DNA, 5819 bp, Artificial Sequence
  Synthetic construct
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgatatcagccatggcagaaccgcgtggcaccagaccagaagaatgatgatgatgatggtgcatatggccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 5 - DNA, 2544 bp, Artificial Sequence
  Synthetic construct
atgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtga

SEQ ID NO: 6 - DNA, 35 bp, Artificial Sequence
  Synthetic construct
aaactcgagtcagagctctggggtaggaagtggtt

SEQ ID NO: 7 - DNA, 32 bp, Artificial Sequence
  Synthetic construct
aaagaattcgacctcaccaaacgctgtctccg

(19) United States
(12) Patent Application Publication
(10) Pub. No.: US 2010/0184146 A1
(43) Pub. Date: Jul. 22, 2010

(54) PLASMID FOR EXPRESSING THIOREDOXIN FUSION PROTEIN AND METHOD FOR PRODUCING TARGET PROTEIN USING SAME

(75) Inventors: Hee-Chul Ahn, Seoul (KR); Hyung Jun Ahn, Seoul (KR); Dong Yun Shin, Seoul (KR); Jong Soo Lim, Seoul (KR); Dae-Ro Ahn, Seoul (KR)

Correspondence Address: SUGHRUE MION, PLLC, 2100 PENNSYLVANIA AVENUE, N.W., SUITE 800, WASHINGTON, DC, 20037, US

(73) Assignee: KOREA INSTITUTE OF SCIENCE AND TECHNOLOGY, Seoul (KR)

(21) Appl. No.: 12/546,367

(22) Filed: Aug. 24, 2009

(30) Foreign Application Priority Data
Jan. 15, 2009 (KR) 10-2009-0003431

(51) Int. Cl. C12P 21/02; C12N 15/70; C12N 1/21; C12P 21/04

(52) U.S. Cl. 435/69.52; 435/320.1; 435/252.33; 435/69.7

(57) ABSTRACT

The present invention provides a plasmid for expressing a thioredoxin fusion protein comprising thioredoxin as a fusion partner, an E. coli cell transformed with the plasmid, and an efficient method for producing a target protein using the E. coli cell. The method according to the present invention can easily remove thioredoxin from thioredoxin fusion protein, so that can produce a target protein of interest in pure and large quantities in the field of medicine and bioengineering.

### FIELD OF THE INVENTION

The present invention relates to a plasmid for expressing a thioredoxin fusion protein, an Escherichia coli cell transformed with the plasmid, and a method for producing a target protein using the E. coli cell.

### BACKGROUND OF THE INVENTION

A variety of fusion partners, e.g., glutathione-S-transferase (Smith, D. B. et al., Gene, 67: 31-40, 1988), maltose-binding protein (Bedouelle, H. et al., Eur. J. Biochem., 171: 541-549, 1988), protein A (Nilsson, B. et al., Mol. Recog., 1: 69-74, 1988) and thioredoxin (Navallie, E. R. et al., Biotechnol., 11: 187-193, 1993), have been widely used for expressing soluble forms of biologically active heterologous proteins in E. coli cells. Thioredoxin, one of the most useful fusion partners, is a protein found in nearly all organisms, which reduces disulfide bonds in proteins into thiols.

Examples of plasmids for expressing thioredoxin fusion proteins include p-BADM-20 (EMBL-Hamburg) having araB promoter site and pET-32 (Novagen) having T7 promoter site, the use of pET-32 plasmid being generally preferred in the art.

The pET-32 plasmid expresses a thioredoxin fusion protein containing an S-tag protein. To selectively obtain a target protein, the thioredoxin fusion protein may be digested with enterokinase, whose cost is, however, high. When such a thioredoxin fusion protein is digested with less expensive thrombin, the target protein may still have the S-tag protein attached thereto, which complicates the structural determination of the target protein. To circumvent this problem, there has been developed a method of placing a gene encoding a protease recognition site in the front of a heterologous protein gene, which comprises inserting a gene encoding protease recognition site in a primer used for PCR amplification of the heterologous protein gene. In this method, the protease recognition site is inserted between the amino acid sequence of the heterologous protein and S-tag coding sequence, to make it possible to separate thioredoxin and S-tag proteins simultaneously using a suitable protease (e.g., TEV protease) for the purpose of obtaining only the target proteins. However, the method is cumbersome in that a protease recognition site must be inserted in a primer every time when a specific protein is to be expressed.

Taking note of the fact that another cleavage enzyme recognition site (i.e., thrombin recognition site) is present in pET-32a plasmid, the present inventors have constructed a novel plasmid from the pET-32a plasmid by deleting S-tag coding sequence via site-specific deletion mutagenesis, and have found that thioredoxin can be easily and selectively generated by treating a fusion protein expressed by said plasmid with thrombin.

### SUMMARY OF THE INVENTION

Accordingly, it is an object of the present invention to provide a plasmid for expressing thioredoxin fusion proteins from which thioredoxin can be easily removed.

It is another object of the present invention to provide a plasmid wherein a gene encoding a target protein is inserted in the multiple cloning site of the plasmid described above.

It is a further object of the present invention to provide an E. coli cell transformed with said plasmids.

It is a still further object of the present invention to provide a method for producing a target protein using the transformed E. coli cell.

In accordance with one aspect of the present invention, there is provided a plasmid which is prepared by deleting the S-tag coding sequence from pET-32a(+) plasmid having the nucleotide sequence of SEQ ID NO:3 represented by the cleavage map of FIG. 1, the pET-32a(+) plasmid comprising a thioredoxin gene, a thrombin recognition site, an S-tag coding sequence, and a multiple cloning site for the insertion of a gene encoding a target protein. Further, there is provided the plasmid having the nucleotide sequence of SEQ ID NO: 4 represented by the cleavage map of FIG. 2, and is designated pET-32-S(−) plasmid.

In accordance with another aspect of the present invention, there is provided the plasmid wherein the gene encoding a target protein is inserted in the multiple cloning site of the pET-32-S(−) plasmid.

In accordance with a further aspect of the present invention, there is provided an E. coli cell transformed with the plasmid.

In accordance with a still further aspect of the present invention, there is provided a method for producing a target protein, comprising i) inserting a gene encoding the target protein in the multiple cloning site of pET-32-S(−) plasmid; ii) transforming E. coli cells with the plasmid obtained in i), and culturing the transformed E. coli cells to express a thioredoxin fusion protein; and iii) treating the thioredoxin fusion protein with thrombin.

### BRIEF DESCRIPTION OF THE DRAWINGS

The above and other objects and features of the present invention will become apparent from the following description of the invention, when taken in conjunction with the accompanying drawings, which respectively show:

FIG. 1: the cleavage map of pET-32a(+) plasmid available from Novagen (MCS: multiple cloning site, trxA: thioredoxin gene, lacI: lac repressor coding gene, Ori: origin for replication, Ap: ampicillin-resistance gene);

FIG. 2: the cleavage map of pET-32-S(−) plasmid prepared by deleting the S-tag sequence from pET-32a(+) plasmid (MCS: multiple cloning site, trxA: thioredoxin gene, lacI: lac repressor coding gene, Ori: origin for replication, Ap: ampicillin-resistance gene);

FIG. 3: a diagram showing bonding pairs between primers used for deleting the S-tag sequence of pET-32a(+) plasmid via site-specific deletion mutagenesis and DNAs of pET-32a(+) plasmid (MCS: multiple cloning site, trxA: thioredoxin gene, lacI: lac repressor coding gene, Ori: origin for replication, Ap: ampicillin-resistance gene); and

FIG. 4: SDS-PAGE analysis results showing the removal of thrombin from a thioredoxin-fused STAT6 SH2 protein which is produced by inserting STAT6 SH2 protein coding sequence into the pET-32-S(−) plasmid and transforming E. coli cells with the plasmid.

### DETAILED DESCRIPTION OF THE INVENTION

Hereinafter, the present invention is described in detail.

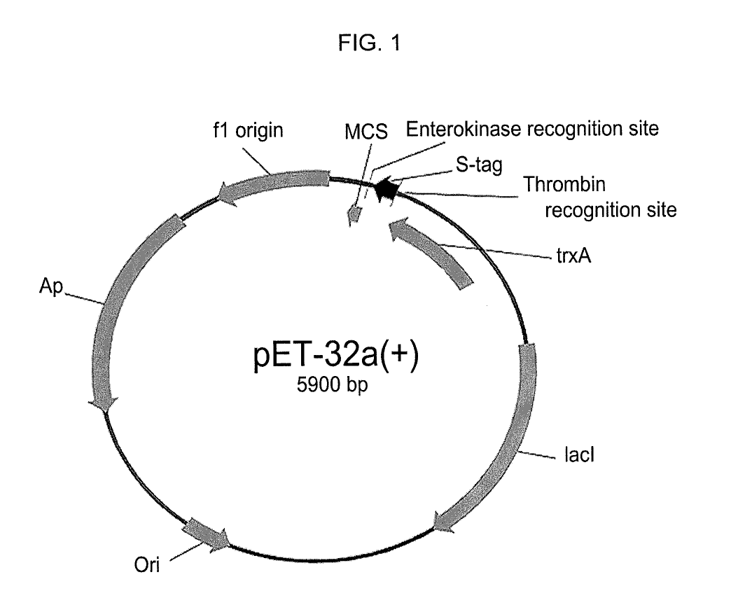

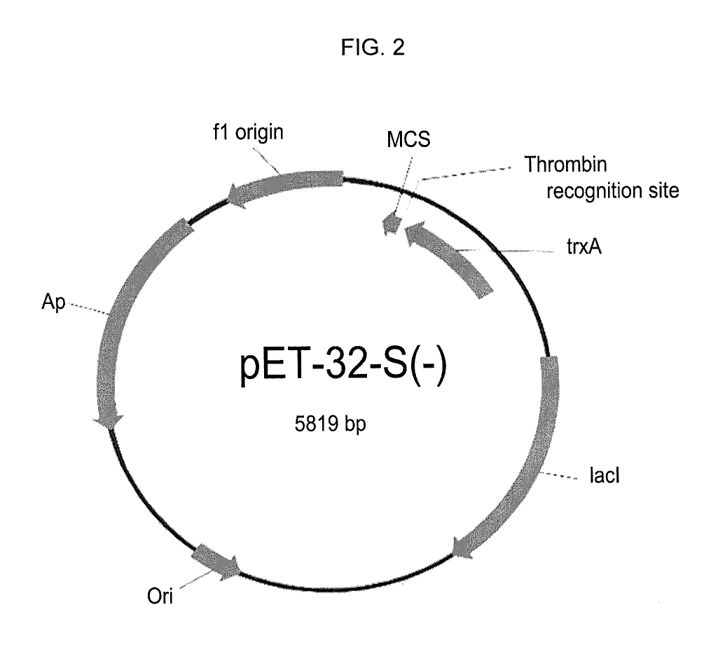

The plasmid of the present invention, which over-expresses a target protein using thioredoxin as a fusion partner, is prepared by deleting the S-tag coding sequence from pET-32a(+) plasmid having the nucleotide sequence of SEQ ID NO: 3 represented by the cleavage map of FIG. 1, the pET-32a(+) plasmid comprising a thioredoxin gene, a thrombin recognition site, an S-tag coding sequence, and a multiple cloning site for the insertion of a gene encoding a target protein. Preferably, the plasmid of the present invention has the nucleotide sequence of SEQ ID NO: 4 represented by the cleavage map of FIG. 2 and is designated pET-32-S(−) plasmid.

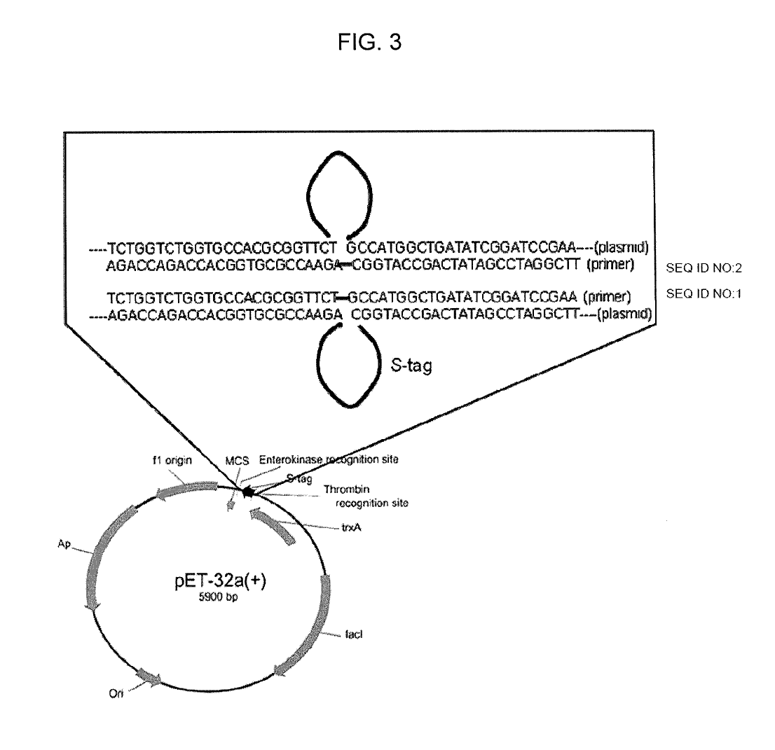

Specifically, the pET-32-S(−) plasmid is characterized by that a thrombin recognition site is inserted between the thioredoxin gene and multiple cloning site, and that the S-tag coding sequence does not exist. The pET-32-S(−) plasmid is prepared by deleting the S-tag coding sequence from the pET-32a(+) plasmid (Novagen; SEQ ID NO: 3) represented by the cleavage map of FIG. 1, through the induction of site-specific deletion mutagenesis via polymerase chain reaction(PCR) using primer pairs of SEQ ID NO: 1 and SEQ ID NO: 2 as shown in FIG. 3.

The plasmid of the present invention comprises T7 promoter for controlling the transcription and expression of a fusion protein as pET-32a(+) plasmid does. In the plasmid of the present invention, the S-tag sequence does not exist and the thrombin recognition site is located in front of the multiple cloning site (5′ side) for the purpose of obtaining only the target protein easily and selectively after the isolation and purification of a fusion protein.

The present invention also provides the plasmid wherein the gene encoding a target protein is inserted in the multiple cloning site of said pET-32-S(−) plasmid.

Said target protein is an active protein which can be easily expressed in E. coli in a soluble form when fused with thioredoxin. The target protein may be selected from the group consisting of human STAT6 SH2 domain, prolyl hydroxylase 2 (PHD2), human β-defensin-2 (hBD2), human interleukin-6 receptor α-chain (hIL-6Rα), mannan-binding lectin-CLR (MBL-CLR), wheat peroxisomal ascorbate peroxidase (pAPX), rhesus macaque interleukin-4(rMamu IL-4), and α-galactosidase.

The plasmid of the present invention may comprise a histidine tag (His-tag) (e.g. six histidine tag), a polyarginine or a consensus biotinylated peptide coding sequence to be linked in the form of translational fusion, at amino terminus and/or carboxyl terminus. Accordingly, the produced fusion protein can be easily isolated and purified using various affinity chromatographies such as nickel chelation column chromatography (e.g. Ni-NTA affinity chromatography).

The present invention also provides an E. coli cell transformed with the plasmid of the present invention. Examples of the E. coli cell include E. coli DH5α, BL21, HB101, JM109 and TH2 etc, but not limited thereto.

The present invention also provides a method for producing a target protein, comprising: i) inserting a gene encoding the target protein in the multiple cloning site of the pET-32-S(−) plasmid; ii) transforming E. coli cells with the plasmid obtained in i), and culturing the transformed E. coli cells to express a thioredoxin fusion protein; and iii) treating the thioredoxin fusion protein with thrombin.

In the method of the present invention, the target protein is an active protein which can be easily expressed in E. coli in a soluble form when fused with thioredoxin, and may be selected from the group consisting of human STAT6 SH2 domain, prolyl hydroxylase 2 (PHD2), human β-defensin-2 (hBD2), human interleukin-6 receptor α-chain (hIL-6Rα), mannan-binding lectin-CLR (MBL-CLR), wheat peroxisomal ascorbate peroxidase (pAPX), rhesus macaque interleukin-4(rMamu IL-4), and α-galactosidase.

The method of the present invention may further comprise steps of isolation and purification of the thioredoxin fusion protein, prior to step (iii). The isolation and purification steps may be carried out using an affinity column chromatography such as nickel chelation column chromatography.

The following Examples are intended to further illustrate the present invention without limiting its scope.

### EXAMPLE 1

### Construction of pET-32-S(−) Plasmid by Site-Specific Deletion Mutagenesis of pET-32a(+) Plasmid

Using a PCR solution composed of pET-32a(+) plasmid of SEQ ID NO: 3 (Novagen) as a template (0.5 μL, 50 ng/μL); primers of SEQ ID NOs: 1 and 2 (in concentration of 1 μL, 10 μL); dNTP mix (1 μL, 100 mM); H₂0 (40 μL); and pfu ultra-polymerase (1 μL), PCR was performed under the following conditions: predenaturation at 95° C. for 5 min; denaturation at 95° C. for 30 sec, annealing at 55° C. for 1 min, followed by 20 cycles of polymerization at 68° C. for 7 min; and final extension at 68° C. for 7 min. The resulting PCR product was digested with restriction enzyme Dpn I at 37° C. for 1 hour. Then, HIT™ competent DH5α cells (Real Biotech) was transformed with the above PCR product, and plasmid DNA was purified therefrom. The sequence analysis of the isolated DNA showed the polynucleotide sequence of SEQ ID NO: 4 (see the structure of FIG. 2), which lacks the S-tag sequence. This plasmid was designated as pET-32-S(−).

### EXAMPLE 2

### Cloning of a Target Gene into pET-32-S(−) and Construction of a Gene Expression Plasmid

STAT6 proteins are phosphorylated by the receptor-associated kinases which are activated by cytokines or growth factors, and play a central role in signal transduction pathway. In order to obtain a sequence encoding SH domain (from 523^{th} to 661^{th} amino acids in STAT6 protein) of STAT6 proteins, using human STAT6 cDNA of SEQ ID NO: 5 (accession number: NM_{—}003153; OriGene Technologies) as a template and primer pair of SEQ ID NOs: 6 and 7, PCR was performed under the following conditions: predenaturation at 95° C. for 5 min; denaturation at 95° C. for 30 sec, annealing at 58° C. for 1 min, followed by 25 cycles of polymerization at 72° C. for 1 min; and final extension at 72° C. for 7 min.

The resulting PCR product was digested with restriction enzymes EcoRI and XhoI, and pET-32-S(−) plasmid obtained in Example 1 was treated with same enzymes. Then, the above PCR product and pET-32-S(−) plasmid were each isolated and purified by electrophoresis on 1% agarose gel, and were ligated each other using T4 DNA ligase at room temperature for 1 hour to obtain a plasmid construct. HIT™ competent DH5α cells (Real Biotech) were transformed with the plasmid construct, followed by isolating the plasmid DNA expressing STAT6 SH2 domain from the transformed cells.

### EXAMPLE 3

### Production and Identification of a Target Protein

E. coli BL21 (DE3) cells were transformed with the plasmid DNA isolated in Example 2. Then, the transformed colonies were inoculated in LB medium (5 mL) containing 50 mg/mL of ampicillin, and cultured overnight at 37° C.

The resulting culture medium was added to 800 mL of LB medium, and expression of protein was induced by addition of IPTG (0.25 mM) when the cell density reached O.D=0.6 at 600 nm. Cells were harvested by centrifugation at 4000 rpm for 15 min using centrifugal separator (Sorvall), and were suspended in 30 mL of PBS buffer solution. Then, lysozyme (100 mg/mL, 300 μL), 2-mercaptoethanol (30 μL) and phenylmethanesulfonylfluoride (PMSF) (100 mM, 150 μL) were added thereto, and the cells were sonicated and centrifuged at 13,000 rpm. From the obtained supernatant, thioredoxin fusion protein was isolated using Ni-NTA (Quiagen) resin. The obtained fusion protein was treated with thrombin, and cultured overnight at room temperature, to remove thioredoxin from the fusion protein. The obtained protein was isolated using fast protein liquid chromatography (FPLC), and was identified by 15% SDS-PAGE and Coomassie blue staining.

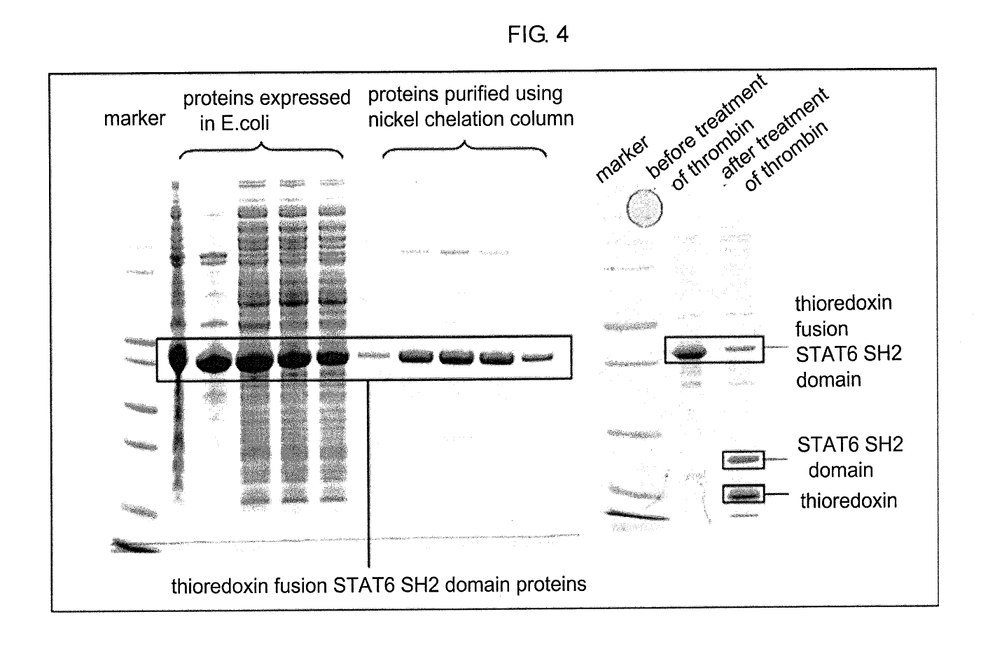

As shown in FIG. 4, it was confirmed that thioredoxin was easily removed by treating thrombin to a thioredoxin fusion protein, STAT6 SH2.

While the invention has been described with respect to the above specific embodiments, it should be recognized that various modifications and changes may be made to the invention by those skilled in the art which also fall within the scope of the invention as defined by the appended claims.

1. A plasmid comprising a thioredoxin gene, a thrombin recognition site, and a multiple cloning site for the insertion of a gene encoding a target protein, wherein the thrombin recognition site is located at 5′ end side of the multiple cloning site and wherein the plasmid has the cleavage map of FIG. 2: and wherein the plasmid lacks S-tag coding sequence.

2. The plasmid of claim 1, which has the nucleotide sequence of SEQ ID NO: 4.

3. The plasmid of claim 21, which further comprises a gene encoding a target protein, said gene being inserted in the multiple cloning site.

4. The plasmid of claim 3, wherein said target protein is selected from the group consisting of human STAT6 SH2 domain, prolyl hydroxylase 2 (PHD2), human β-defensin-2 (hBD2), human interleukin-6 receptor α-chain (hIL-6Rα), mannan-binding lectin-CLR (MBL-CLR), wheat peroxisomal ascorbate peroxidase (pAPX), rhesus macaque interleukin-4 (rMamu IL-4), and α-galactosidase.

5. An E. coli cell transformed with the plasmid according to claim 3.

6. The E. coli cell of claim 5, which is selected from the group consisting of DH5α, BL21, HB101, JM109, and TH2.

7. A method for producing a target protein, comprising:
i) inserting a gene encoding the target protein in the multiple cloning site of the plasmid of claim 2;
ii) transforming E. coli cells with the plasmid obtained in i), and culturing the transformed E. coli cells to express a thioredoxin fusion protein;
iii) treating the thioredoxin fusion protein with thrombin to produce the target protein.

8. The method of claim 7, wherein the target protein is selected from the group consisting of human STAT6 SH2 domain, prolyl hydroxylase 2(PHD2), human β-defensin-2 (hBD2), human interleukin-6 receptor α-chain (hIL-6Rα), mannan-binding lectin-CLR (MBL-CLR), wheat peroxisomal ascorbate peroxidase (pAPX), rhesus macaque interleukin-4 (rMamu IL-4), and α-galactosidase.

9. The method of claim 7, which further comprises steps of isolation and purification of the thioredoxin fusion protein prior to step (iii).
